(19) United States
(12) Patent Application Publication
(10) Pub. No.: US 2016/0362717 A1
(43) Pub. Date: Dec. 15, 2016

(54) COMPOSITION FOR CONTAMINANT IDENTIFICATION OF COSMETICS AND CONTAINER FOR COSMETICS COMPRISING THE SAME

(71) Applicant: Do Jeong KWON, Busan (KR)

(72) Inventor: Do Jeong KWON, Busan (KR)

(21) Appl. No.: 15/178,036

(22) Filed: Jun. 9, 2016

(30) Foreign Application Priority Data
Jun. 9, 2015 (KR) 10-2015-0081425

(51) Int. Cl. C12Q 1/04; C12Q 1/10

(52) U.S. Cl. CPC C12Q 1/04; C12Q 1/10

(57) ABSTRACT

A composition for contaminant identification of cosmetics and a container for cosmetics capable of indicating contamination of cosmetics provided with the composition for contaminant identification of cosmetics are provided, wherein the composition for contaminant identification of cosmetics including an aqueous solution dissolving anthocyanin to have absorbance of 0.15 to 1 when employing distilled water as an absorbance control and measuring absorbance in a range of 510 nm to 720 nm; a beef extract; NaCl; lactose; glucose; and agar powder. The composition for contaminant identification of cosmetics is formed with substances harmless to the skin and indicates a degree of contamination of cosmetics. Accordingly, when a container for cosmetics provided with such a composition for contaminant identification is manufactured, color changes in the composition for contaminant identification of cosmetics can be visually identified, and therefore, consumers, distributors, and the like can easily identify contamination of cosmetics.

### CROSS-REFERENCE TO RELATED APPLICATIONS

This application claims the benefit of and priority to Korean Patent Application No. 10-2015-0081425, filed on Jun. 9, 2015, in the Korean Intellectual Property Office, the disclosure of which is incorporated herein by reference in its entirety.

### BACKGROUND

### 1. Field

Embodiments of the present disclosure relates to a composition for contaminant identification of cosmetics and a container for cosmetics including the same, and in particular, to a composition visually indicating the contamination of cosmetics and a container for cosmetics including the same, thereby indicating the contamination of cosmetics included in the container.

### 2. Description of Related Art

Cosmetics are highly valued products that have been used for human beauty care from as far back as ancient times. Such cosmetics are classified into various types based on their function, and are delivered to and consumed by consumers through diverse methods. Currently, research into performance enhancements in wrinkle improvement, skin whitening, and moisturizing cosmetics have been steadily carried out within the cosmetics industry; however, specific measures against contamination that may occur during the distribution of cosmetics or during use by consumers have not been taken.

Some modern day cosmetics are made from natural ingredients, and therefore, contamination is more likely to occur, and rapid contamination and acidification progression from air, light, contact with heat, and, in particular, secondary contamination by hands after cosmetics have been opened, is more likely to occur. Specifically, when cosmetics are contaminated by bacteria, a pH value of the cosmetics may be changed due to metabolites excreted with bacterial growth. Consequently, awareness of the importance of cosmetics management has increased; however, no obvious solutions have thus far been found.

In particular, in the case of cosmetics, contamination rapidly progresses when it becomes impossible to limit bacteria with preservative substances due to frequent opening and contact with hands. Due to the nature of the products, cosmetics are in contact with hands at least once a day after being opened, and if the hands are not suitably clean, the rate of contamination is further accelerated.

Examples of methods for preventing such contamination of cosmetics may include a method of using a vacuum container to fully seal off air contact, or a method of mixing antibiotics with cosmetics so as to create a difficult environment in which bacteria can grow. However, the first method is disadvantageous in terms of technological and economic aspects, and the second method may cause skin irritation, which is contradictory to the purpose of using cosmetics.

As described above, contamination prevention is not a simple matter, and therefore, it is preferable to at least identify contamination of cosmetics so as to prevent the usage of contaminated cosmetics.

Accordingly, providing a composition for contaminant identification of cosmetics harmless to the human body and directly visually indicating a state of contamination of cosmetics, and providing a container for cosmetics provided with such a composition, are expected to be useful in the related art.

### SUMMARY

An aspect of the present disclosure may provide a composition harmless to the skin and indicating contamination of cosmetics.

Another aspect of the present disclosure may provide a container for cosmetics provided with the composition for contaminant identification of cosmetics described above.

One embodiment of the present disclosure may provide a composition for contaminant identification of cosmetics including an aqueous solution dissolving anthocyanin to have absorbance of 0.15 to 1 when employing distilled water as an absorbance control and measuring absorbance in a range of 510 nm to 720 nm; a beef extract; NaCl; lactose; glucose; and agar powder, wherein the composition includes the beef extract in 0.01% by weight to 2% by weight, the NaCl in 0.001% by weight to 0.1% by weight, the lactose in 0.1% by weight to 10% by weight, the glucose in 0.001% by weight to 0.1% by weight, the agar powder in 1% by weight to 10% by weight and a remainder of the anthocyanin-dissolved aqueous solution.

The anthocyanin-dissolved aqueous solution is preferably an extract of a plant selected from the group consisting of cabbage leaves, black beans, rose petals, and morning glory petals.

Another embodiment of the present disclosure may provide a container for cosmetics indicating contamination of cosmetics including at least one contamination indicating unit formed as a transparent or semi-transparent concave portion inside the container for cosmetics, wherein the composition for contaminant identification of cosmetics of the present disclosure is provided in the contamination indicating unit.

A total area of the contamination indicating unit is preferably from 5% to 50% of a total area of the container for cosmetics.

The contamination indicating unit is preferably formed at a depth of 0.1 cm to 0.5 cm in the container for cosmetics.

A contamination indicating layer formed inside the contamination indicating unit is preferably formed at a thickness of 0.05 cm to 0.3 cm.

### BRIEF DESCRIPTION OF DRAWINGS

The above and other aspects, features, and advantages of the present disclosure will be more clearly understood from the following detailed description taken in conjunction with the accompanying drawings, in which:

FIGS. 1A and 1B illustrate a container for cosmetics of one embodiment of the present disclosure, and FIG. 1C illustrates a cross-section based on A-A′ in FIG. 1B;

FIGS. 2A, 2B, and 2C identify possibility of contamination on a surface of cosmetics;

FIGS. 3A and 3B identify possibility of contamination within cosmetics;

FIGS. 4A, 4B and 4C show pictures of different bacteria grown in a composition for contaminant identification of cosmetics according to one embodiment of the present disclosure affecting a color of a medium. FIGS. 4A and 4B show media after culture, and FIG. 4C shows a medium prior to culture;

FIGS. 5A and 5B show one example of a container for cosmetics including a composition for contaminant identification of cosmetics of one embodiment of the present disclosure. FIG. 5A shows a front view and FIG. 5B shows a plane view;

FIGS. 6A, 6B, and 6C show a process of an illustrative container for cosmetics of FIGS. 5A and 5B, indicating contamination of cosmetics over progression of time; and

FIG. 7 summarizes an aspect of an anthocyanin color change by pH.

### DETAILED DESCRIPTION

Hereinafter, embodiments in the present disclosure will be described in detail with reference to the accompanying drawings. The disclosure may, however, be embodied in many different forms and should not be construed as being limited to the embodiments set forth herein. Rather, these embodiments are provided so that this disclosure will be thorough and complete, and will fully convey the scope of the disclosure to those skilled in the art. In the drawings, the shapes and dimensions of elements may be exaggerated for clarity, and the same reference numerals will be used throughout to designate the same or like elements.

According to an embodiment in the present disclosure, a composition for contaminant identification of cosmetics indicating a degree of contamination of cosmetics is provided, and in particular, the composition for contaminant identification of cosmetics according to an embodiment of the present disclosure includes an aqueous solution dissolving anthocyanin to have absorbance of 0.15 to 1 when employing distilled water as an absorbance control and measuring absorbance in a range of 510 nm to 720 nm, a beef extract, NaCl; lactose, glucose, and agar powder. As described above, distilled water is employed as an absorbance control in the present disclosure.

Herein, absorbance represents a ratio of absorbed light intensity with respect to incident light intensity when parallel rays penetrate one medium, and is unitless.

The composition for contaminant identification of cosmetics according to an embodiment of the present disclosure uses only natural substances and is harmless to the skin, and the presence of bacteria proliferated from contamination by hands may be identified from pH value changes in the cosmetics caused by metabolites excreted with bacterial growth.

The composition for contaminant identification of cosmetics according to an embodiment of the present disclosure preferably includes the beef extract in 0.01% by weight to 2% by weight, the NaCl in 0.001% by weight to 0.1% by weight, the lactose in 0.1% by weight to 10% by weight, the glucose in 0.001% by weight to 0.1% by weight, the agar powder in 1% by weight to 10% by weight based on the aqueous solution dissolving anthocyanin to have absorbance of 0.15 to 1 when employing distilled water as an absorbance control and measuring absorbance in a range of 510 nm to 720 nm, and a remainder of the anthocyanin-dissolved aqueous solution.

The composition for contaminant identification of cosmetics according to an embodiment of the present disclosure more preferably includes the beef extract in 0.05% by weight to 0.1% by weight, the NaCl in 0.002% by weight to 0.008% by weight, the lactose in 0.5% by weight to 5% by weight, the glucose in 0.002% by weight to 0.008% by weight, the agar powder in 0.5% by weight to 5% by weight based on the aqueous solution dissolving anthocyanin to have absorbance of 0.15 to 1 when employing distilled water as an absorbance control and measuring absorbance in a range of 510 nm to 720 nm, and a remainder of the anthocyanin-dissolved aqueous solution.

When the anthocyanin absorbance measured in a range of 510 nm to 720 nm is less than 0.15 in the anthocyanin-dissolved aqueous solution, there is a problem in that visual identification of pH value changes in the cosmetics caused by metabolites excreted with bacterial growth is not insufficient, and when the anthocyanin absorbance is greater than 1, such substances may leak into the cosmetics composition.

When the beef extract is included in less than 0.01% by weight based on the anthocyanin aqueous solution, reflection on the color changes may not be sufficient, since cell death is induced by an oligotrophic state. When the beef extract is included in greater than 2% by weight based on the anthocyanin aqueous solution, possibility of propagation of unexpected bacteria exists, and substances may leak to be mixed with cosmetics. Typical bacteria grown using such a beef extract include E. coli, but are not limited thereto.

When the NaCl is included in less than 0.001% by weight based on the anthocyanin aqueous solution, reflection on the color changes may not be sufficient, since individual bacterium is destroyed by a storage solution. Meanwhile, when the NaCl is included in greater than 0.1% by weight based on the anthocyanin aqueous solution, a phenomenon of moisture of cosmetics being absorbed to a medium may occur by an osmosis phenomenon.

When the lactose is included in less than 0.1% by weight based on the anthocyanin aqueous solution, reflection on the color changes may not be sufficient, since cell death is induced by an oligotrophic state. Meanwhile, when the lactose is included in greater than 10% by weight based on the anthocyanin aqueous solution, propagation of bacteria may be caused from an oversupply of nutrients.

When the glucose is included in less than 0.001% by weight based on the anthocyanin aqueous solution, reflection on the color changes may not be sufficient, since cell death is induced by an oligotrophic state. Meanwhile, when the glucose is included in greater than 0.1% by weight based on the anthocyanin aqueous solution, propagation of bacteria may be caused from an oversupply of nutrients.

In the present disclosure, the agar powder is a material providing a role for indication by fixing bacteria generated from contamination, and when the agar powder is included in less than 1% by weight based on the anthocyanin aqueous solution, viscosity of the composition for contaminant identification of cosmetics of the present disclosure decreases, causing a problem of increasing the amount of the composition mixed with cosmetics, and when the agar powder is included in greater than 10% by weight based on the anthocyanin aqueous solution, the gel structure is excessively dense, leading to reduced moisture migration, and a disturbance in smooth color changes may occur.

Moreover, the composition for contaminant identification of cosmetics of the present disclosure includes a remainder of the anthocyanin-dissolved aqueous solution.

In the present invention, the anthocyanin-dissolved aqueous solution may particularly use naturally occurring plant extracts, and for example, may use extracts of plants selected from the group consisting of cabbages, black beans, roses, blueberries, and morning glory petals. More specifically, at least one extract may be selected from among the extracts obtained by extracting cabbage leaves, beans of black beans, rose petals, morning glory petals, and blueberries.

Cabbage leaf extract obtained as above includes a large quantity of anthocyanin, and therefore, is suitable to be used in the composition for contaminant identification of cosmetics of the present disclosure.

The composition for contaminant identification of cosmetics according to an embodiment of the present disclosure indicates pH value changes in cosmetics, and pH value changes in any range from pH 0 to pH 13 may be identified based on a pH value when the cosmetics are manufactured.

If the pH value is closer to 0, the composition for contaminant identification of cosmetics of the present disclosure may be red, and as the pH value becomes higher, the color may become white, yellow, and light orange after changing from violet, purple, and blue.

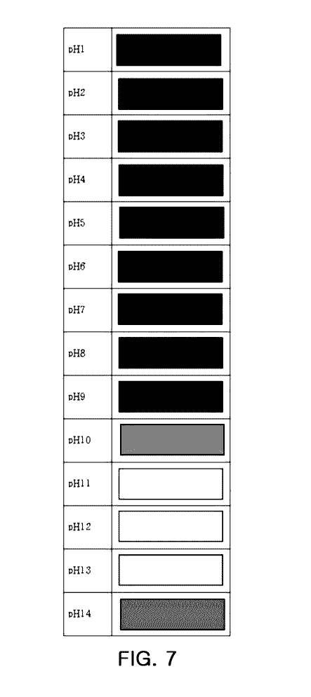

FIG. 7 summarizes an aspect of an anthocyanin color change by a pH value. However, the color identified by the naked eye may be somewhat different depending on the anthocyanin content, and other cosmetics compositions such as the beef extract, the NaCl, the lactose, the glucose, the agar, and other substances.

Meanwhile, microorganisms identified by the composition of the present disclosure are not particularly limited as long as they affect a pH value, and examples thereof may include at least one microorganism selected from the group consisting of Microbacterium testaceum, Curtobacterium sp., Pantoea eucrina partial, Arthrobacter crystallopoietes, Stenotrophomonas chelatiphaga, Novosphingobium resinovorum, Microbacterium phyllosphaerae, Bacillus cereus, Bacillus megaterium, Enterobacterludwigii, Lysinibacillus sphaericus, Lysinibacillus fusiformis, and Bacillus thuringiensis.

As another embodiment, the composition for contaminant identification of cosmetics of the present disclosure may be prepared as follows.

For example, one of the naturally occurring plant extracts, preferably a cabbage extract, may be obtained by introducing a red cabbage leaf edge with distilled water in an amount of 10 parts by weight to 15 parts by weight per 100 parts by weight of the distilled water, and heating the result for 5 to 20 minutes over a medium-low heat. The corresponding extract is filtered using, for example, Whatman® filter paper, Korean handmade paper, another type of filter paper, and the like, to obtain an anthocyanin-dissolved aqueous solution in which solids are removed. Through such a process, each extract may be obtained by mixing a naturally occurring plant extract, for example, a plant selected from the group consisting of black beans, roses, blueberries, and morning glory petals with distilled water in an amount of 10 parts by weight to 15 parts by weight per 100 parts by weight of the distilled water, and then heating the result for 5 minutes to 20 minutes over a medium-low heat, and an anthocyanin-dissolved aqueous solution in which solids are removed may be obtained by filtering the result using a Whatman® filter paper, Korean handmade paper, another type of filter paper, and the like.

To the anthocyanin-dissolved aqueous solution obtained as above, the lactose, the glucose, the beef extract, the NaCl, and the agar powder are mixed within the content ranges of the present disclosure, and herein, the mixture is sufficiently mixed through stirring so as not to produce lumps. Subsequently, sterilization is performed using a moist sterilizer such as an autoclave.

The sterilization is performed at 110° C. to 140° C., preferably at 115° C. to 130° C., and more preferably at 120° C. to 125° C. and maintained for 5 to 30 minutes, preferably for 10 to 20 minutes, and more preferably for approximately 15 minutes.

Furthermore, another embodiment of the present disclosure may provide a container for cosmetics easily indicating contamination of cosmetics by being provided with a contamination indicating unit formed with the composition for contaminant identification of cosmetics described above.

Specifically, the container for cosmetics indicating contamination of cosmetics according to an embodiment of the present disclosure may include at least one contamination indicating unit formed with a transparent or semi-transparent concave portion inside the container for cosmetics, and the composition for contaminant identification of cosmetics of the present disclosure is provided in the concave portion formed inside the contamination indicating unit. The contamination indicating unit is preferably transparent or semi-transparent, and more preferably transparent since color changes need to be identified from an external viewpoint.

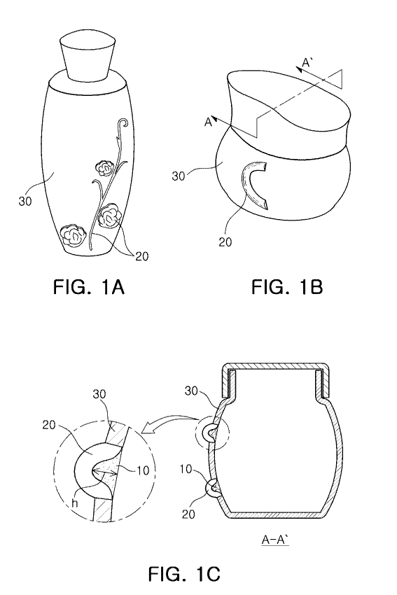

FIGS. 1A and 1B illustrate the container for cosmetics of an embodiment of the present disclosure. FIG. 1C shows a cross-section based on A-A′ in FIG. 1B, and includes a section of the contamination indicating unit formed with a transparent or semi-transparent concave portion (20). The part of the container other than the contamination indicating unit (30) is shown as being opaque; however, transparency of this part may be properly adjusted as desired. The composition for contaminant identification of cosmetics (10) described above is provided in the concave portion formed inside the contamination indicating unit.

Herein, in the contamination indicating unit, a thickness of the corresponding part of the container for cosmetics protrudes while having the same thickness as the part of the container other than the contamination indicating unit, and as a result, either a concave portion may be obtained inside the container and a convex portion outside the container, or a concave portion may be obtained as a thickness of the container decreases with the inner wall side of the container caving in without forming a convex portion outside the container.

Meanwhile, the form of the contamination indicating unit is not particularly limited as long as identification from an external viewpoint is possible with the composition for contaminant identification being provided therein, and the form may be obtained as many separate shapes as shown in FIG. 1A, or may be obtained as one figure as shown in FIG. 1B, and the shapes are not particularly limited.

Preferably, the form may be one or more narrow and long shapes crossing an up and down direction or a left and right direction of the side surface of the container for cosmetics, or may have a form of many small figures being distributed on the whole surface of the container, and this has an advantage in that contamination identification is possible throughout the entire container.

The total area of the contamination indicating unit is preferably from 5% to 50% of the total area of the container for cosmetics, and if the total area of the contamination indicating unit is less than 5% of the area of the container for cosmetics, there is a problem in that the degree of overall cosmetic contamination is not sufficiently reflected, and the area being greater than 50% is not preferable since the composition for contaminant identification provided inside the contamination indicating unit is excessively consumed, which increases production costs, and may cause visual repulsion.

Meanwhile, the contamination indicating unit is preferably formed at a depth of 0.1 cm to 0.5 cm when the part of the container other than the cosmetic contamination indicating unit (30) is employed as a container base surface. For example, as shown in FIG. 1C, the depth of the contamination indicating unit (h) is preferably from 0.1 cm to 0.5 cm, and the depth of less than 0.1 cm causes a problem of the composition for contaminant identification being not sufficiently provided in the concave portion formed inside the contamination indicating unit, and the depth of greater than 0.5 cm may cause handling difficulties when the container is formed of glass and the like, or may cause concern for damage due to a reduced thickness.

Herein, a contamination indicating layer formed inside the contamination indicating unit is formed inside the concave portion at a thickness of preferably 0.05 cm to 0.3 cm, and more preferably 0.1 cm to 0.3 cm. For example, as shown in FIG. 1C, the contamination indicating layer may be provided in a form of filling the whole concave portion formed inside the container of the contamination indicating unit, or may be formed as an alternative certain layer when the contamination indicating unit has a large area; however, the formation is not limited thereto.

Hereinafter, the present disclosure will be described in more detail with reference to specific examples. However, the following examples are for illustrative purposes only, and the scope of the present disclosure is not limited thereto.

### EXAMPLE

1. Identification of Possible Cosmetic Contamination by Hand

(1) Implementation of Contamination Possibility on Cosmetic Surface

Using an EC agar medium, a medium with no inoculation (A), a medium touched by a finger (B), and a medium smearing an essence (Time Revolution Night Repair, Missha®) with a hand (C) were cultured for a week at 24° C. to 25° C., that is, at room temperature.

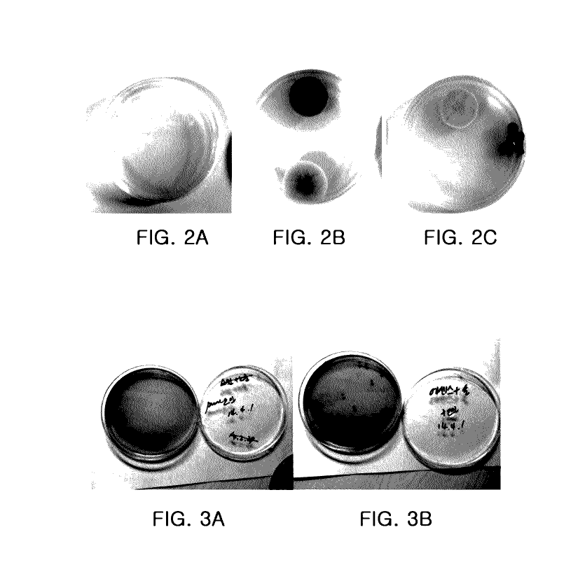

As a result, it was identified that, a colony was formed in FIGS. 2B and 2C, and surroundings where the colony was formed were discolored. Herein, the rate of colony expression was the highest in FIG. 2C.

(2) Implementation of Contamination Possibility Inside Cosmetics

Oil was boiled to a high temperature to be sterilized, then cooled and poured on a medium (A) inoculated with a sample (Time Revolution Night Repair, Missha®) to form an anaerobic environment. Meanwhile, oil was also poured on a medium (B) on which a sample (Time Revolution Night Repair, Missha®) was inoculated and then smeared with a hand (finger), to form an anaerobic environment. Subsequently, the results were cultured for a week at 24° C. to 25° C., that is, at room temperature.

As a result, it was identified that, in the medium (A), sterilization of the oil was well accomplished, since no microorganisms grew; whereas, in the medium (B), a colony was formed, and it was identified that proliferation of bacteria is possible within cosmetics even under an anaerobic environment, as shown in FIGS. 3A and 3B.

2. Preparation of Composition for Contaminant Identification of Cosmetics According to Present Disclosure

A cabbage was introduced with purified water in an amount corresponding to 10% by weight to 15% by weight of the purified water, and the result was heated for 15 minutes over a medium-low heat. After the heating, cabbage remnants produced during the heating process were filtered using Whatman® filter paper, Korean handmade paper, another type of filter, paper and the like.

1 L of the cabbage extract obtained as above and the substances of the following Table 1 were mixed. The mixing order was not limited; however, mixing was sufficiently carried out so as to completely dissolve all the substances in order to prepare the composition for contaminant identification of cosmetics of the present disclosure. Herein, the composition was purple.

TABLE 1

| | | |
|---|---|---|
| Cabbage Extract | 1 | L |
| Lactose | 10 | g |
| Beef Extract | 0.67 | g |
| NaCl | 0.05 | g |
| Glucose | 0.05 | g |
| Agar | 15 | g |

3. Identification of Bacteria Capable of Being Identified by Composition for Contaminant Identification of Cosmetics According to Present Disclosure

On the composition for contaminant identification of the present disclosure prepared in the above-described 2, the following 8 cosmetics were smeared with a finger, and the results were stored for 3 days at room temperature.

TABLE 2

| | Sample Cosmetics Manufacturer (Name of Cosmetics) |
|---|---|
| 1 | Laneige ® (Water Sleeping Pack) |
| 2 | Tony Moly ® (Intense Care Snail Cream) |
| 3 | Missha ® (Ultra Waterfull Clear Cream) |
| 4 | Mary Kay ® (Intense Moisturizing Cream) |
| 5 | Hera ® (Cell Essence (Cell-Bio Fluid Sync)) |
| 6 | Biotherm ® (Aqua Source Hydration Cream) |
| 7 | Lancome ® (Hydra Zen Neurocalm Extreme Soothing |
| | Moisturizing Cream Gel) |
| 8 | SK2 ® (Two-Facial Treatment Clear Lotion) |

As a result, as shown in the following Table 3, a colony changing the composition for contaminant identification of the present disclosure from purple to red or blue was identified, and herein, the composition for contaminant identification of the present disclosure changing from purple to red or blue means either a pH value decrease or a pH value increase. Accordingly, bacteria and fungi able to proliferate in each cosmetic and identifiable by the composition of the present disclosure were identified as shown in the following Table 3.

Only manufacturing companies of the cosmetics are listed in the following Table 3, and specific names of the cosmetics are as listed in Table 2.

TABLE 3

| | Name of Bacteria | Cosmetics | Medium Color Change |
|---|---|---|---|
| 1 | Bacillus thuringiensis | Lancome | Purple > Bluish Green |
| 2 | Bacillus sp. | Laneige | Purple > Bluish Green |
| 3 | Curtobacterium sp. | Lancome | Purple > Yellow |
| 4 | Pantoea eucrina | Lancome | Purple > Red |
| 5 | Arthrobacter | Laneige | Purple > Bluish Green |
| | crystallopoietes | | |
| 6 | Microbacterium | Biotherm | Purple > Bluish Green |
| | phyllosphaerae | | |
| 7 | Bacillus cereus | SK2 | Purple > Bluish Green |
| 8 | Bacillus meqaterium | SK2 | Purple > Bluish Green |
| 9 | Enterobacter ludwiqii | SK2 | Purple > Bluish Green |

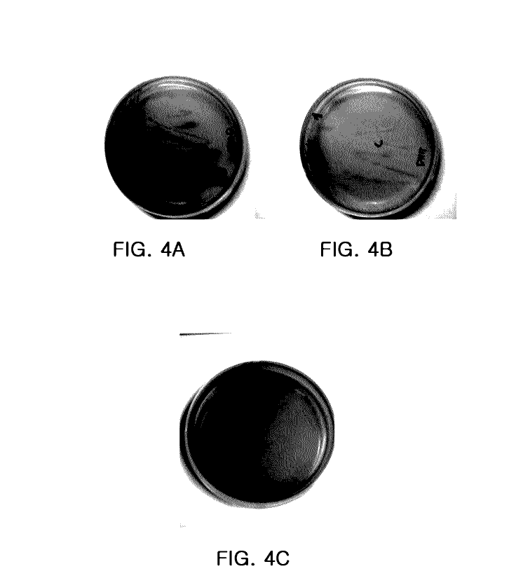

The pictures of FIGS. 4A, 4B, and 4C (the following pictures) show different bacteria grown in the composition for contaminant identification of cosmetics of the present disclosure obtained in the above-described 2 affecting the color of the medium. FIG. 4A is a picture of DH20 after culture and FIG. 4B is a picture of DH7 after culture. When these were compared with FIG. 4C, which shows a medium prior to culture, it was identified that the color of the medium had changed.

In a medium growing bacteria in a cosmetic in a non-contaminated state as a control group of the test, it was identified that only one fungus (mold) colony was formed in each ‘Lancome: Hydra Zen Neurocalm Extreme Soothing Moisturizing Cream Gel’ and ‘Laneige: Water Sleeping Pack’, and other bacteria did not grow. Accordingly, it was seen that the colony growing in the test was contaminated bacteria, rather than bacteria having been present in the cosmetics.

4. Preparation of Container for Cosmetics Including Composition for Contaminant Identification of Cosmetics

(1) Manufacturing Process of Container

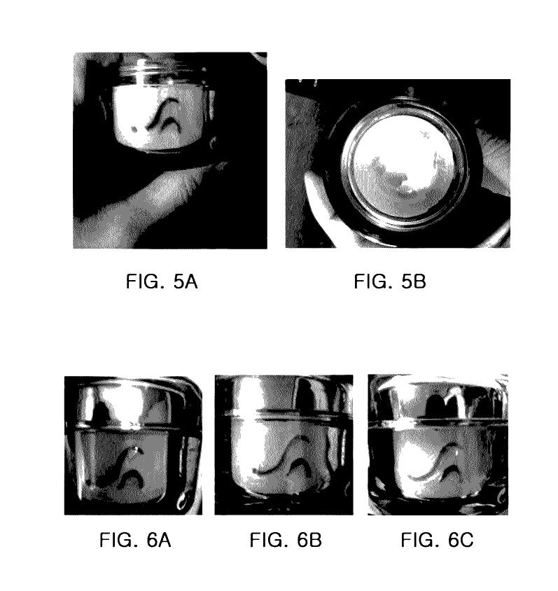

Beeswax was poured into an existing glass container for cosmetics with a diameter of approximately 6 cm and a height of 4 cm, and was thoroughly cooled. The cooled beeswax was removed from the container, and a space to hold FIG. 5B and cosmetic contents was made by digging, and a wall surface of the beeswax was formed at a thickness of 0.5 cm. Subsequently, the wall surface of the beeswax was dug in the shape such as FIG. 5A and penetrated. The inside of the penetrated shape was filled with the composition for contaminant identification of the present disclosure prepared in the above-described 2, and after the composition hardened, Hydra Zen Neurocalm Extreme Soothing Moisturizing Cream Gel by Lancome, which had a pH of 7, was placed as cosmetic contents in empty space of the beeswax, and the progress was observed.

(2) Identification of Color Changes in the Composition for Contaminant Identification

In the concave portion of the container for cosmetics manufactured in (1), the composition for contaminant identification of cosmetics of the present disclosure was placed as shown in the pictures of FIGS. 5A and 5B, and the color of the composition for contaminant identification immediately after placing the cosmetics inside the container was blue.

Pictures were taken at an interval of 30 days, and the test was carried out in a manner of touching the cosmetics with a finger twice a day, in the morning and evening. After 90 days had passed, it was identified that all the colors had changed to almost completely pink. FIG. 6A is a picture taken after 30 days, FIG. 6B is a picture taken after 60 days, and FIG. 6C is a picture taken after 90 days.

The cosmetics used in the test had a pH of 7 prior to contamination; however, it was identified that the pH value changed to 5.20 after the test, and it was also identified that the color of the composition for contaminant identification of cosmetics of the present disclosure changed from blue to pink.

According to the present disclosure, a composition for contaminant identification of cosmetics formed with substances harmless to the skin, and indicating a degree of contamination of cosmetics, may be obtained. Accordingly, when manufacturing a container for cosmetics provided with such a composition for contaminant identification, color changes in the composition for contaminant identification of cosmetics can be visually identified, and therefore, contamination of cosmetics can be readily checked by consumers, distributors, and the like.

While embodiments have been shown and described above, it will be apparent to those skilled in the art that modifications and variations could be made without departing from the scope of the present disclosure as defined by the appended claims.

What is claimed is:

1. A composition for contaminant identification of cosmetics, the composition comprising:
an aqueous solution dissolving anthocyanin to have absorbance of 0.15 to 1 when employing distilled water as an absorbance control and measuring absorbance in a range of 510 nm to 720 nm;
a beef extract;
sodium chloride;
lactose;
glucose; and
agar powder,
wherein the composition includes the beef extract in 0.01% by weight to 2% by weight, the sodium chloride in 0.001% by weight to 0.1% by weight, the lactose in 0.1% by weight to 10% by weight, the glucose in 0.001% by weight to 0.1% by weight, the agar powder in 1% by weight to 10% by weight, and a remainder of the anthocyanin-dissolved aqueous solution.

2. The composition for contaminant identification of cosmetics of claim 1, wherein the anthocyanin-dissolved aqueous solution comprises an extract of a plant selected from the group consisting of cabbage leaves, black beans, rose petals, and morning glory petals.

3. A container for cosmetics configured to indicate contamination of cosmetics comprising at least one contamination indicating unit formed as a transparent or semi-transparent concave portion inside the container for cosmetics, wherein the composition for contaminant identification of cosmetics of any one of claim 1 or 2 is provided in the contamination indicating unit.

4. The container for cosmetics capable of indicating contamination of cosmetics of claim 3, wherein a total area of the contamination indicating unit is from 5% to 50% of a total area of the container for cosmetics.

5. The container for cosmetics capable of indicating contamination of cosmetics of claim 3, wherein the contamination indicating unit is formed at a depth of 0.1 cm to 0.5 cm from the container for cosmetics.

6. The container for cosmetics capable of indicating contamination of cosmetics of claim 3, wherein a contamination indicating layer formed inside the contamination indicating unit is formed at a thickness of 0.05 cm to 0.3 cm.
